(19) United States
(12) Patent Application Publication
(10) Pub. No.: US 2006/0089685 A1
(43) Pub. Date: Apr. 27, 2006

(54) Chemiluminescent phototherapy device

(75) Inventors: Douglas J. Streibich, Clayton, NY (US); Robert S. Healy, Clayton, NY (US)

Correspondence Address: HISCOCK & BARCLAY, LLP, 2000 HSBC PLAZA, ROCHESTER, NY, 14604-2404, US

(73) Assignee: HealthShine Inc.

(21) Appl. No.: 11/058,021

(22) Filed: Feb. 15, 2005

Related U.S. Application Data
(60) Provisional application No. 60/621,851, filed on Oct. 25, 2004.

(51) Int. Cl. A61N 5/06

(52) U.S. Cl. 607/88

(57) ABSTRACT

A portable, disposable, and self-contained medical device for the production and delivery of therapeutic light to the blood transmitted through the non-ocular skin surface. The light is generated by a chemiluminescent dye configured to radiate light in the specific wavelength required by a particular phototherapy treatment. The wavelength of light emitted by this planar device is such that some disorders of health requiring phototherapy are effectively treated. Disorders falling into this category include elevated bilirubin jaundice, circadian rhythm anomalies, mood disorders and potentially a reduction in injury or surgically related bruise healing time.

### CROSS-REFERENCE TO RELATED APPLICATION

This application claims priority to U.S. Provisional Patent application No. 60/621,851, filed Oct. 25, 2004.

### FIELD OF THE INVENTION

This invention relates to methods and apparatus for medical treatment, and more particularly to the treatment of phototherapy-treatable disorders, such as jaundice.

### BACKGROUND OF THE INVENTION

Hyperbilirubinemia jaundice) is common in infants, and affects, in some degree, up to 50% of full-term infants, and most preterm infants. Bilirubin is the end result of chemical reactions involved in the breakdown of hemoglobin molecules. Bilirubin circulates through the blood stream chiefly in unconjugated form, and is processed by catalysis in the liver for conversion into a water-soluble form, which can then be excreted into the intestines as bile. The livers of newborn infants tend to have limited ability to process bilirubin, so infants are prone to accumulation of unconjugated bilirubin, and thus develop jaundice. In most cases, the jaundice is mild, and resolves spontaneously during the first week of life. However, jaundice is potentially dangerous, as high levels of bilirubin are toxic to brain tissue.

While the immaturity of liver cells is the chief cause of jaundice, there may be pathologic causes, which include hemolytic anemia, polycythemia, extravasated blood, and even metabolic disorders. These pathologic causes can create sudden and severe onset of excess bilirubin levels. The goal of medical intervention is to mitigate or curtail the rise in bilirubin levels in the blood, to avoid a toxic accumulation. Approximately 10% of newborns require such intervention.

It is well known that when infants are exposed to light in the violet and blue regions of the spectrum (particularly wavelengths of 430 to 470 nanometers or nm), a photochemical reaction takes place in the skin. The photochemical reaction changes unconjugated bilirubin into more soluble metabolites, including photobilirubin, which is then excreted into the bile, and if further photooxidation occurs prior to excretion, generates products which are excreted in the urine. Such phototherapy has proven to be an effective treatment for the vast majority of infants with unconjugated hyperbilirubinemia.

Infant phototherapy for jaundice is generally administered by phototherapy units, the effectiveness of which depend, at least in part, on the irradiance delivered by the light source, and the amount of skin exposed to the light. The light delivery systems in common use in hospital settings fall into two general categories, the first of which involves a crib-like structure for holding the infant, surmounted by banks of fluorescent or halogen lamps. These systems deliver light in the abovementioned violet and blue regions of the spectrum, at the target intensity of 5 to 9-μW/cm²/nm of bandwidth. This type of phototherapy unit has a number of disadvantages. First, the target light intensity is at a level at which retinal damage is of concern, and consequently the infant must wear protective eye patches. Secondly, to maximize the area exposed to the phototherapy, the infants must be essentially naked; since such infants have difficulty in temperature regulation, they must be maintained in temperature-controlled isolettes during phototherapy. Maintenance in temperature-controlled isolettes, in turn, tends to reduce the availability of human contact. The bulk and cost of the isolettes, in turn, tends to limit the use of this first type of phototherapy unit to hospital environments.

A second type of phototherapy unit which is generally available is the fiber-optic phototherapy blanket. This is a relatively flexible panel-like support for holding the ends of the fibers of one or more fiber-optic cables adjacent to a surface of the blanket, so that light propagating through the optical fibers is directed toward one side of the panel. This phototherapy blanket can be placed on the bottom of a conventional isolette, so that the infant can be illuminated from the bottom, as well as from the top by fluorescent or halogen lamps conventionally disposed. For infants with milder degrees of hyperbilirubinemia, the fiber-optic phototherapy blanket may be used alone, by wrapping the flexible panel about the infant's body, and securing the panel in place. Since the panel is opaque, there is less concern that the light can affect the infant's eyes, which tends to reduce the need for eye protection. If the panel is wrapped about the torso, the child can be dressed over the panel to keep it warm, and thus attains at least some mobility, which allows parental interaction, albeit limited by the optical fiber cable tethered to the light source. Since such phototherapy blanket units are relatively compact, they are more amenable to home use than the more conventional phototherapy cribs. The ability to provide home therapy for mild cases of jaundice tends to reduce healthcare costs by eliminating the need for hospitalization in all but severe cases of jaundice.

The fiber-optic phototherapy blankets, while significant improvements over the crib-type units, have some disadvantages. For instance, patients are treated only over limited regions of the body, such as the torso. Also, while the infant may be treated at home, the infant is effectively fixed in location to a selected room, because of the short length of the fiber-optic cable, and because of the need for a powerful light source for driving the light-source end of the fiber-optic cable, which, in turn, requires access to the AC power mains. Yet further, the halogen bulbs ordinarily used to drive fiber-optic cables have a life rated at 1000 hours, which is about 40 days, and the bulbs have a cost near $40.00 each.

A further type of phototherapy unit is a garment with a flexible support material shaped so as to be worn adjacent to the skin of at least a portion of the wearer's body. The garment is in the shape of an infant jumpsuit, gown, shirt, or blanket. A plurality of semiconductor or solid-state light sources is affixed to the support material of the garment in such a manner that, when energized, each of the light sources radiates toward the skin of the wearer's body. In the context of a blanket-shaped garment, the light sources radiate from one of the two principal broad surfaces of the blanket. An energization coupler is connected to the light sources, for coupling a power source such as batteries or a fuel cell to the light sources. In response to the electrical energization coupled to the light sources, the skin of the wearer of the garment is illuminated by the light sources. The solid-state light source removes the need for an external light source, however an external power supply is still required, whether it comprises batteries, a fuel cell, or AC power.

There are a number of patents that teach such prior phototherapy methods, such as U.S. Pat. No. 6,045,575 to Rosen, et al. (Rosen). Rosen teaches an apparatus for treating neonatal jaundice in the form of a garment, which has semiconductor light sources affixed thereto for radiating toward the inside of the garment when the infant is dressed in the garment. A portable energy source such as batteries or a fuel cell powers the array of light sources. Thus Rosen has the disadvantage of requiring a separate power source.

U.S. Pat. No. 4,761,047 to Mori teaches a light rays radiation cloth for medical treatment with a flexible basic sheet plate, a large number of optical fibers arranged on one surface of the basic sheet plate, and a light rays connector for supplying light rays to the optical fibers. The optical fibers each have a light rays emitting portion for emitting the light rays from one surface of the basic sheet plate. Mori's apparatus has the disadvantage of requiring an external light source, an optical conductor cable, and a power source.

U.S. Pat. No. 3,877,437 to Maitan, et al. (Maitan) teaches an apparatus for simultaneous bilateral phototherapy (pan irradiation) of infants stricken with neonatal jaundice. Maitan's apparatus requires that the infant be placed in an entirely transparent chamber and two groups of light sources placed respectively one above and one below the chamber. The light sources taught by Maitan are fluorescent bulbs. Thus Maitan teaches a phototherapy device with the disadvantages of an external light source, which requires a power supply, a special transparent chamber and bed to contain the infant, and protection for the infant's eyes. Further, such devices require that the infant be substantially naked and thus require careful temperature control.

Therefore, a phototherapy device that does not require an external light source and thus does not require a special transparent chamber or an optical conductor cable is desired in the art.

Further, a phototherapy device that does not require a power supply is desired in the art.

Even further, a phototherapy device that does not require protection for the patient's eyes is desired in the art.

Still further, a phototherapy device that allows the patient, particularly an infant, to be clothed thus easing the control of the ambient temperature is desired in the art.

Still further, a phototherapy device that generates substantially no heat and therefore has no risk of burning the patient is desired in the art.

### SUMMARY OF THE INVENTION

The invention comprises, in one form thereof, a phototherapy light source for treating bilirubin jaundice having a chemiluminescent dye that emits radiation in the wavelengths of about 430 nanometers to about 470 nanometers and a reservoir for the chemiluminescent dye with at least one panel that is transparent from about 430 nanometers to about 470 nanometers and is made of a physiologically safe material for contact with infants. The phototherapy light source further includes an activator chemical and the chemiluminescent dye is in solution with an activator compliment catalyst. The reservoir is a flexible bladder, which includes a plurality of ribs that connect opposing walls of the bladder. Each of the ribs defines a plurality of fluid bypass passageways. The activator chemical is contained in a chamber within the bladder and the chamber is configured to rupture when a significant pressure is applied to it. A reflective inner surface may be affixed to or integral with the bladder and the bladder may be affixed to an inner surface of a garment. Alternatively, the reservoir is a substantially rigid and transparent cylinder.

Additionally, the invention comprises a phototherapy device for treating an ailment, including a sealed container, a solution having a chemiluminescent dye within the container, and a sealed chamber containing an activator chemical, wherein said chamber is operable to release the activator into the solution. The solution further includes an activator compliment catalyst. The chemiluminescent dye is configured to emit light substantially within a range of wavelengths particularly suited to treat the ailment, wherein the range of wavelengths is about 430-nm to about 470-nm. The container is a flexible bladder having a plurality of ribs that connect opposing walls of the container. Each of the ribs defines a plurality of fluid bypass passageways. The chamber is configured to rupture when a significant pressure is applied to said chamber.

Further, the invention comprises a phototherapy process for treating an ailment. The process includes the steps of sealing a solution having a chemiluminescent dye in a container and sealing an activator chemical in a chamber within the container, wherein the chamber is operable to release the activator to mix with the solution. The process includes the further steps of activating the chemiluminescent dye and placing the container in close proximity to an area affected by the ailment to expose the affected area to a range of light rays emitted by the activated chemiluminescent dye. The container is discarded after the affected area has been treated for a prescribed period of time. The processes is particularly useful for treating ailments such as hyperbilirubinemia (jaundice), in which case the range of light wavelengths is about 430-nm to about 470-nm.

An advantage of the present invention is that the phototherapy device comprises a self-contained light source and has a high degree of flexibility so as not to reduce or inhibit normal body motion. The modular form of the structural architecture allows for multiple configurations in the shape of the apparatus, from small planar strips for infants to large surface patches for adult applications. No special transparent chambers are needed to contain the patient.

A further advantage of the present invention is that it is not tethered to an energy source for the production of therapeutic light, nor does it require bulky battery packs. The portability of the invention allows users to maintain normal activities

An even further advantage of the present invention is that the phototherapy device allows the user to conceal the apparatus underneath garments so that the patient's eyes are shielded from the light and temperature control is less of an issue.

A still further advantage of the present invention is that there is no risk of heat burn as no noticeable heat is generated by the chemiluminescent process

A still further advantage of the present invention is that the device is disposable and made in such a way as to minimize cost of production and thus be available to a wider range of economic users.

### BRIEF DESCRIPTION OF THE DRAWINGS

The above-mentioned and other features and advantages of this invention, and the manner of attaining them, will become apparent and be better understood by reference to the following description of several embodiments of the invention in conjunction with the accompanying drawings, wherein:

FIG. 1 is an isometric view of one embodiment of the phototherapy device of the present invention;

FIG. 2 is a top view of the device shown in FIG. 1;

FIG. 3 is a side view of the device shown in FIG. 1;

FIG. 4 is an end view of the device shown in FIG. 1;

FIG. 5 is a an isometric view of an alternative configuration of the device shown in FIG. 1; and

FIG. 6 is an isometric view of a second embodiment of the phototherapy device of the present invention.

Corresponding reference characters indicate corresponding parts throughout the several views. The examples set out herein illustrate several embodiments of the invention but should not be construed as limiting the scope of the invention in any manner.

### DETAILED DESCRIPTION

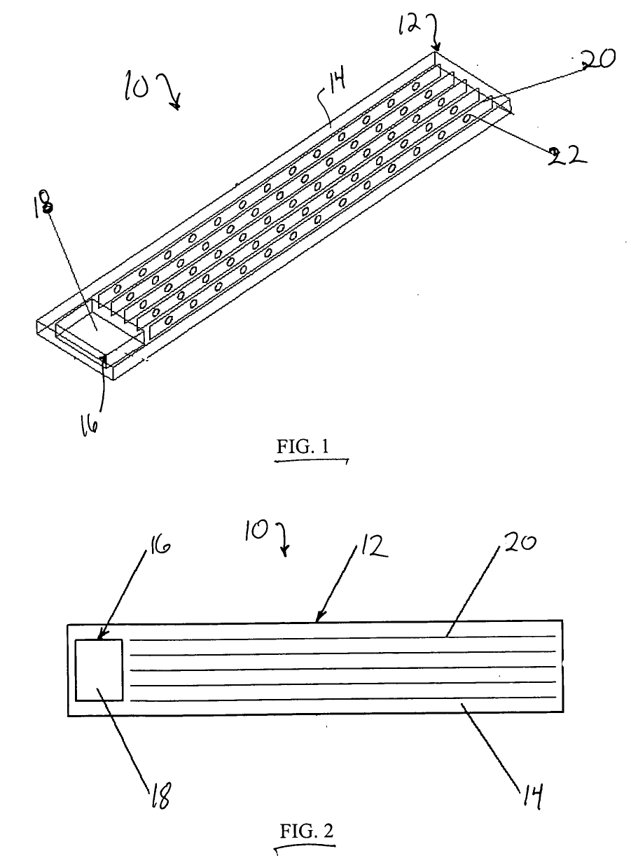

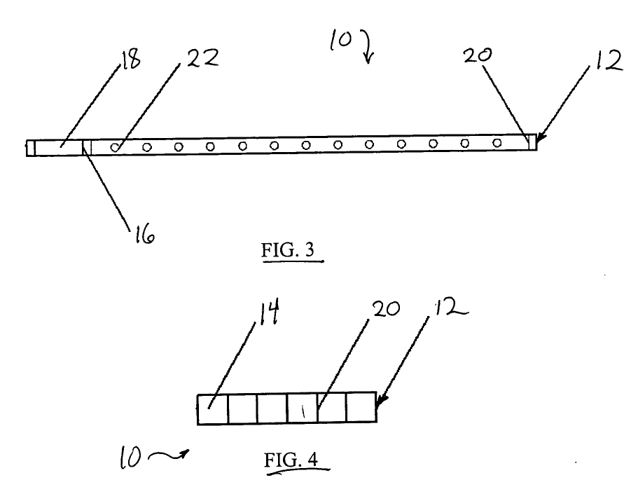

Referring to FIGS. 1-4, there is shown the phototherapy device according to one embodiment of the present invention. The phototherapy device 10 includes a container 12, a dye solution 14, a chamber 16, and an activator solution 18.

According to the present embodiment, the container 12 is a disposable, flexible bladder made of a material approved for use by the U.S. Food and Drug Administration (FDA). The container 12 is substantially transparent or translucent and includes a plurality of ribs 20, each of which have a plurality of fluid bypasses 22. The ribs 20 provide structural support for the container 12 and maintain the planar shape of the container 12. The fluid bypasses 22 allow the dye solution 14, which is sealed within the container, to pass through the ribs 20. Further, space is provided between the ends of the ribs 20 and the end walls of the container 12 to thereby allow the dye solution 14 to communicate through this space and distribute the pressure in the container 12. This has the advantage of reducing the chances of fluid being trapped between ribs and forming a pressure pocket that could rupture the container 12. A further advantage of the rib configuration of the container 12 is that the dye, and therefore the light, is distributed throughout the container 12.

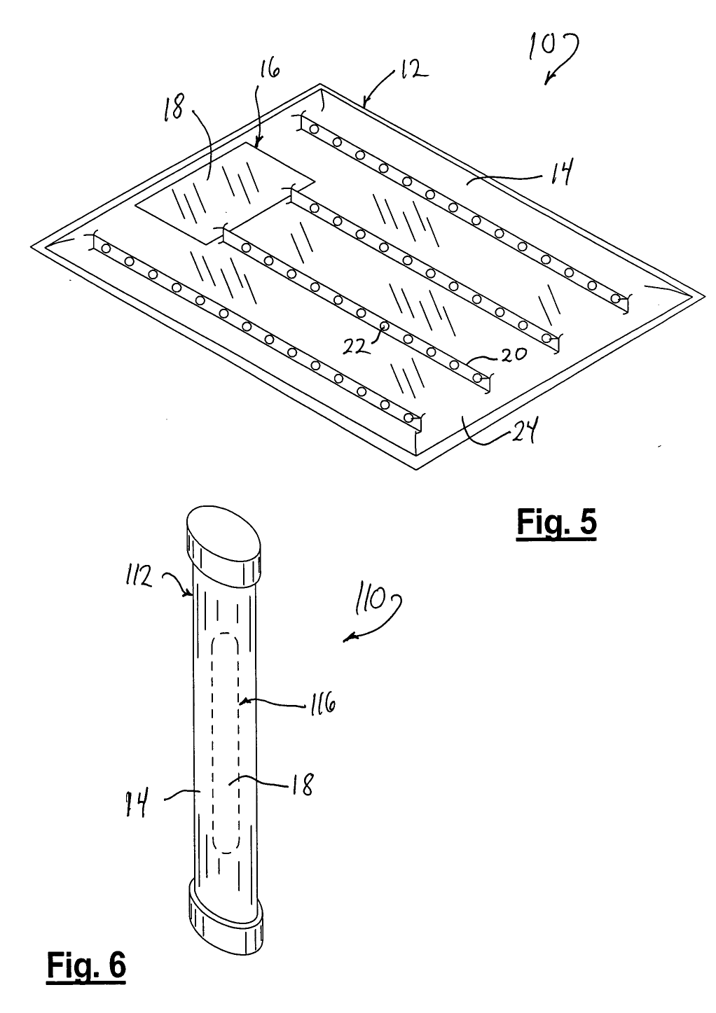

Alternative to the shape shown in FIGS. 1-4, the container 12 may have any planar shape, such as circular and square shapes. In the current embodiment, however, the thickness of the container 12 is less than 25% of its width and the length of the container 12 is greater than or equal to its width. An alternative configuration of the container 12 is shown in FIG. 5. In FIG. 5, the walls of the container 12 are welded to form a flange around its perimeter.

The container 12, as shown in FIG. 5, optionally includes a reflective surface 24, such as Mylar, on one wall to direct the majority of the light emitted by the phototherapy device 10 through one side of the device. Further, the phototherapy device 10 may be incorporated into a blanket or garment, in which case, protective eyewear may not be needed because the garment may keep the light from reaching the eyes. The container 12 optionally is affixed to the blanket or garment with an adhesive. Alternatively, the container 12 is in the form of a bandage or patch affixed directly to the affected area using an adhesive. Such a bandage may be particularly useful for treating contusions or lacerations. The bandage may include an absorbent portion in conjunction with the light emitting portion to help control bleeding.

The size of the planar container 12 is determined by the application. For example, a blanket-sized container 12 is used to treat an infant for jaundice so that phototherapy may be provided to as much as the infant's skin as possible. In a further example for treating contusions or lacerations, a bandage that is large enough to cover the bruised or wounded area is used to provide phototherapy only to the localized area.

The dye solution 14 is an aqueous solution containing a chemiluminescent dye such as Bis(2,4,6-trichlorophenyl)oxalate in the presence of 9,10-diphenylanthracene (as a fluorescer), which emits light in wavelengths that peak between 430-nm to 470-nm, and is particularly suited for treating hyperbilirubinemia. Alternatively, other chemiluminescent dyes may be used in the invention individually or in combination in order to produce light in any range of wavelengths required for treating a particular ailment. The possible ranges of wavelengths include those outside the visible spectrum, such as ultraviolet and infrared. The solution further includes a stable solvent, which maintains constituent properties. For example, benzyl acetate or benzyl benzoate may be used as the stable solvent in the dye solution 14. The dye solution 14 also contains an activator compliment catalyst such as sodium salicylate.

In the present embodiment, the dye solution 14 does not completely fill the container 12 so that the container 12 is not under significant pressure during normal use. The volume of each application shall be such that an effective therapeutic levels of light shall be produced. In determining the volume of the solutions, one must consider the treatment period required, the intensity of the light output from the activated dye solution 14, the transparency of the container 12, and the efficiency of the reflector material 24, if included.

The chamber 16 is a sealed pouch containing the activator solution 18. The chamber 16 is sealed within the container 12 and the ribs 20 retain the chamber 16 in one end of the container 12. The chamber 16 is optionally affixed to a wall of the container 12. The chamber 16 keeps the activator solution 18 separate from the dye solution 14, and is configured to rupture when pressure is applied to the chamber 16 to thereby allow the activator solution 18 to mix with the dye solution 14.

The activator solution 18 is an aqueous solution containing an activator such as peroxide containing compounds, including but not limited to hydrogen peroxide producing components.

In use, the phototherapy device 10 is assembled by a manufacturer. The ribs 20 are affixed to the walls of the container 12 such as by thermal welding. The walls of the container 12, except for the end containing the chamber 16, are sealed so that the ribs 20 do not contact each other. The dye solution 14 is placed in the container 12 and the activator solution 18 is sealed in the chamber 16. The chamber 16 is placed in the container 12 and the container 12 is sealed closed. The sealing is carried out by any method suitable for the container material, such as thermal or ultrasonic welding for certain types of plastics.

The end user applies pressure to the chamber 16 until it ruptures and releases the activator solution 18 into the dye solution 14. The user kneads or shakes the container 12 to mix the activator solution 18 and the dye solution 14. The resulting chemical reaction between the activator chemical and the activator compliment catalyst adds energy to the chemiluminescent die to thereby cause the electrons in the dye atoms to elevate to a higher energy level. As the electrons in the dye atoms fall to their normal energy levels, they release energy in the form of light.

Once the chemiluminescent dye is activated, the user places the phototherapy device next to the area to be treated, such as skin affected by hyperbilirubinemia, so that the light given off by the chemiluminescent dye is directed towards the affected area. The phototherapy device 10 may be placed under clothing or a blanket thereby reducing the need for fine ambient temperature control. Once the affected area has been treated for the prescribed period of time, or the reaction slows and the dye ceases to emit light, the user discards the used phototherapy device 10. In the case that the treatment time is less than the life of the phototherapy device 10, the user may place the phototherapy device 10 in a cold place such as a freezer to slow the reaction. When the phototherapy device 10 is warmed to room temperature for a subsequent treatment period, the reaction will resume its normal rate and the chemiluminescent dye will emit light until the reaction is completed.

An additional ingredient optionally included in the dye solution 14 acts as an indicator, which allows for the visualization of useful process termination. This may include time-released fluorescers or multi-unstable/stable fluorescers. More particularly, a time release system according to the present embodiment consists of peroxide unstable packets having coatings, such as oxide decaying cellulose, that dissolve and release their contents at a predetermined time following exposure to the peroxide activator 18. The contents of the packets may be powdered or aqueous dye that will be passively diffused about the chamber. The color of the indicator dye is a color that contrasts with the color of the therapeutic dye in the present embodiment. For example, a red indicator dye might be used with a blue therapeutic dye. In an indicator dye system using multi-unstable/stable fluorescers, an unstable fluorescer, such as 5,12 Bis-Phenylethynyl Napthacene is used in conjunction with a stable fluorescer such that the phototherapy device glows a different color, such as red, when the therapeutic dye is spent. Further description of the use of unstable fluorescers is included in U.S. Pat. No. 6,267,914 to Cranor, which is herein incorporated by reference. In either indicator dye system, the end user is instructed to remove the phototherapy device 10 when it changes color and replace it with a new device 10 as required by the prescribed treatment regimen.

It should be particularly noted that since chemiluminescent devices generally do not produce heat, there is no risk of overheating or burning the patient. Further, the phototherapy device 10 is inexpensive to produce and simple to use. Since the phototherapy device 10 does not require an external power source or batteries, the device is portable and non-restricting to thereby allow the patient to maintain normal activities.

It should be noted that only U.S. FDA-approved materials are used in the present embodiment. It should also be noted that an alternative activator compliment catalyst used in the invention is phenyl oxalate ester. It should be further noted that, in one embodiment, the activator solution 18 includes between about 2 to 5 molar hydrogen peroxide and the dye solution 14 includes about 0.75 percent by weight fluorescer.

In an alternative embodiment, shown in FIG. 6, the phototherapy device 110 includes a container 112 and a chamber 116. The container 112 is a substantially rigid vessel that contains the dye solution 14. The chamber 116 is relatively brittle and contains the activator chemical 18. Bending of the container 112 breaks the chamber 116 to release the activator chemical 18 into the dye solution 14. The activator chemical 118 reacts with the activator compliment catalyst to activate the chemiluminescent dye, as described above.

In a further alternative embodiment, the dye solution is in the form of a balm that may be applied to the affected area. In use, the activator chemical is mixed with an amount of the balm to activate the chemiluminescent dye, and then the balm is applied to the affected area. In this embodiment, the activator chemical, the activator compliment catalyst, and any products resulting from the reaction between them do not react with the affected area, such as skin, or any substances that may be on the affected area to avoid undesirable reactions.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A phototherapy light source for treating bilirubin jaundice, comprising:
a chemiluminescent dye that emits radiation in the wavelengths of about 430 nanometers to about 470 nanometers; and
a reservoir for said chemiluminescent dye with at least one panel that is transparent from about 430 nanometers to about 470 nanometers and is made of a physiologically safe material for contact with infants.

2. The phototherapy light source of claim 1, further comprising an activator chemical and wherein said chemiluminescent dye is in solution with an activator compliment catalyst.

3. The phototherapy light source of claim 2, wherein said reservoir comprises a flexible bladder for containing said solution, and wherein said bladder comprises a plurality of ribs that connect opposing walls of the bladder.

4. The phototherapy device of claim 3, wherein each of the ribs defines a plurality of fluid bypass passageways.

5. The phototherapy device of claim 3, wherein said activator chemical is contained in a chamber within said bladder, wherein said chamber is configured to rupture when a significant pressure is applied to said chamber.

6. The phototherapy device of claim 3, wherein said bladder comprises a wall having a reflective inner surface.

7. The phototherapy device of claim 3, wherein said bladder is affixed to an inner surface of a garment.

8. The phototherapy device of claim 2, wherein said reservoir is a substantially rigid and transparent cylinder.

9. The phototherapy device of claim 1, further comprising an indicator dye that is noticeable when said chemiluminescent dye is spent, wherein said indicator dye has a different color than said chemiluminescent dye.

10. A phototherapy device for treating an ailment, comprising:
a) a sealed container;
b) a solution having a chemiluminescent dye within said container;
c) a sealed chamber containing an activator chemical, wherein said chamber is operable to release the activator into said solution; and
d) an activator compliment catalyst in said solution.

11. The phototherapy device of claim 10, wherein the chemiluminescent dye is configured to emit light substantially within a range of wavelengths particularly suited to treat said ailment.

12. The phototherapy device of claim 11, wherein the range of wavelengths is about 430 nanometers to about 470 nanometers.

13. The phototherapy device of claim 10, wherein said container is a substantially rigid and transparent cylinder.

14. The phototherapy device of claim 10, wherein said container is a flexible bladder having a plurality of ribs that connect opposing walls of the container, and wherein each of the ribs defines a plurality of fluid bypass passageways.

15. The phototherapy device of claim 10, wherein said container comprises a wall having a reflective inner surface.

16. The phototherapy device of claim 10, wherein said container is affixed to an inner surface of a garment.

17. A phototherapy process for treating an ailment, comprising:
a) sealing a solution having a chemiluminescent dye in a container;
b) activating the chemiluminescent dye; and
c) placing the container in close proximity to an area affected by said ailment to expose the affected area to a range of light rays emitted by the activated chemiluminescent dye.

18. The phototherapy process of claim 17, wherein the range of light wavelengths is best suited for treating said ailment.

19. The phototherapy process of claim 17, further comprising the step of discarding the container after the affected area has been treated for a prescribed period of time.

20. The phototherapy process of claim 17, further comprising the step of sealing an activator chemical in a chamber within the container.

21. The phototherapy process of claim 20, wherein said activating step comprises rupturing the chamber to release the activator into the solution.

22. The phototherapy process of claim 21, wherein the solution contains an activator compliment catalyst that reacts with the activator chemical to add energy to the chemiluminescent dye.

23. The phototherapy process of claim 17, wherein the container comprises a flexible bladder having a plurality of ribs that connect opposing walls of the container, and wherein each of the ribs forms a plurality of fluid bypass passageways.

24. The phototherapy process of claim 17, further comprising the step of affixing the container to an inner surface of a garment.

25. The phototherapy process of claim 17, wherein said ailment is hyperbilirubinemia and the range of light wavelengths is about 430 nanometers to about 470 nanometers.
